(19) United States
(12) Patent Application Publication
(10) Pub. No.: US 2024/0177331 A1
(43) Pub. Date: May 30, 2024

(54) COMPUTER-BASED POSTURE ASSESSMENT AND CORRECTION

(71) Applicant: Microsoft Technology Licensing, LLC, Redmond, WA (US)

(72) Inventor: Mastafa Hamza FOUFA, Nimes (FR)

(73) Assignee: Microsoft Technology Licensing, LLC, Redmond, WA (US)

(21) Appl. No.: 18/059,365

(22) Filed: Nov. 28, 2022

(51) Int. Cl. G06T 7/70; G06F 3/16; G06T 5/50; G06T 7/20; G06T 17/00; G06V 10/70

(52) U.S. Cl. CPC G06T 7/70; G06F 3/16; G06T 5/50; G06T 7/20; G06T 17/00; G06V 10/70; G06T 2207/20221; G06T 2207/30196

(57) ABSTRACT

Examples are disclosed that relate to performing computer-based assessment of a human subject's posture and providing computer-based posture correction. In one example, a computing system comprises a posture assessment machine and a posture correction machine. The posture assessment machine receives posture assessment signals from posture assessment sensors and outputs an assessment of a human subject's posture based on the posture assessment signals. The posture assessment signals include images of a human subject. The posture correction machine receives the images of the human subject and the assessment of the human subject's posture, generates a virtual clone of the human subject having an improved posture relative to the human subject's posture, and generates a composite image including the virtual clone admixed with an image of the human subject.

### BACKGROUND

As more people spend long hours sitting in front of computers on a regular basis, the risk of poor posture increases significantly. Standing is a good alternative to sitting for a long period of time. However, even when standing a person can have poor posture. Either sitting or standing with poor posture for a long period of time can negatively affect a person's wellbeing, collaboration with other people, and overall productivity.

### SUMMARY

Examples are disclosed that relate to performing a computer-based assessment of a human subject's posture and providing computer-based posture correction to improve the human subject's posture. In one example, a computing system comprises a posture assessment machine and a posture correction machine. The posture assessment machine receives one or more posture assessment signals from one or more posture assessment sensors and outputs an assessment of a human subject's posture based at least on the one or more posture assessment signals. The one or more posture assessment signals include one or more images of a human subject. The posture correction machine receives the one or more images of the human subject and the assessment of the human subject's posture, generates a virtual clone of the human subject having an improved posture relative to the human subject's posture, and generates a composite image including the virtual clone admixed with an image of the human subject.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 shows an example scenario in which a human subject is interacting with a computer, and the computer assesses the human subject's posture and provides posture correction.

FIG. 2 shows an example computing system that is configured to assess a human subject's posture and provide posture correction.

FIG. 3 shows example posture assessment sensors that output posture assessment signals for assessing a human subject's posture.

FIG. 4 shows an example composite image including an image of a human subject admixed with a virtual clone of the human subject having an improved posture relative to the human subject's posture.

FIG. 5 shows an example composite image including posture adjustment feedback indicating whether a human subject's posture approaches an improved posture of a virtual clone.

FIG. 6 shows an example posture assessment notification including a plurality of images of a human subject captured over a posture tracking duration.

FIG. 7 shows an example posture assessment notification including a visual representation of a human subject's posture during different time intervals.

FIGS. 8-10 show different example posture assessment notifications.

FIG. 11 shows an example computer-implemented method for assessing and correcting a human subject's posture.

FIG. 12 shows an example computer-implemented method for progressively assessing a human subject's posture over a posture tracking duration.

FIG. 13 shows an example computing system.

### DETAILED DESCRIPTION

The present description is directed to a computer-based approach for assessing a human subject's posture and performing posture correction in order to proactively improve the human subject's posture. In particular, a posture assessment artificial intelligence (AI) machine receives posture assessment signals from posture assessment sensors and outputs posture assessments based at least on the posture assessment signals. Further, a posture correction AI machine receives the posture assessment signals and the assessment of the human subject's posture and outputs posture correction feedback based at least on the posture assessment signals and the assessment of the human subject's posture. Such posture correction feedback brings about awareness of the human subject's posture and helps the human subject to improve their posture.

In some implementations, the posture correction AI machine outputs instantaneous posture correction feedback in the form of a virtual clone of the human subject having an improved posture relative to the human subject's assessed posture. The posture correction AI machine generates the virtual clone of the human subject from image(s) of the human subject. As used herein, the term “virtual clone of a human subject” generally represents a virtual avatar having an appearance that corresponds to the appearance of the human subject. Further, the posture correction AI machine generates a composite image that includes the virtual clone admixed with an image of the human subject, so that the human subject can make posture correcting adjustments that approach the improved posture of the virtual clone. By generating the composite image including both the human subject and the virtual clone, the human subject is provided with a visual comparison that the human subject can use to correct the human subject's posture. The composite image is referred to as instantaneous posture correction feedback, because it provides a current snapshot that the human subject can react to in real time to adjust their posture.

In some implementations, the posture correction AI machine progressively updates the assessment of the human subject's posture over a posture tracking duration and provides progressive posture correction feedback in the form of a posture assessment notification that visually summarizes how the human subject's posture changes over the posture tracking duration. By providing a visual summary of how the human subject's posture changes over the posture tracking duration, the human subject is able to recognize a distinct change in the human subject's posture and make adjustments as needed to improve their posture.

Such an approach provides the technical benefit of improving human computer interaction by assessing and correcting a human subject's posture while the human subject interacts with a computer. Such improved posture can positively affect the human subject's wellbeing, collaboration with other people, and overall productivity.

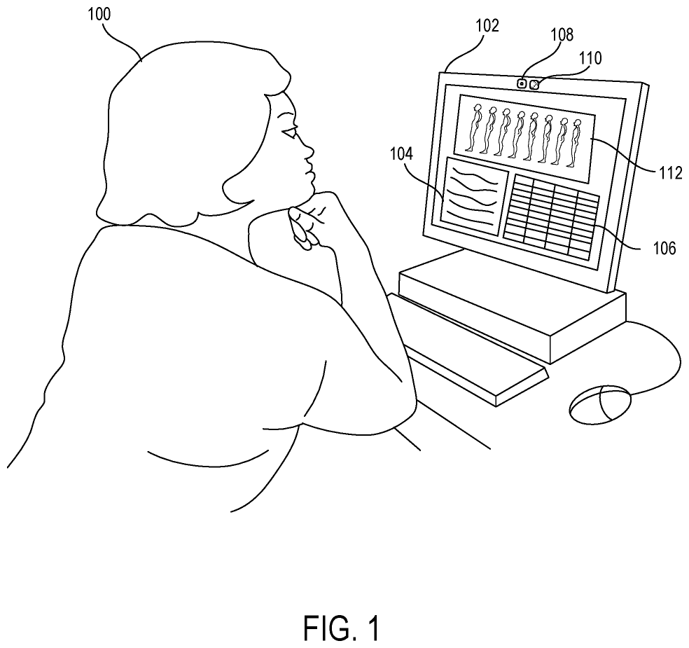

FIG. 1 shows an example scenario where a human subject 100 is interacting with a computer 102. The computer 102 executes a plurality of computer application programs. For example, the human subject 100 is having a conversation with another person via a personal communication application program in the form of an instant messenger application program 104. At the same time, the human subject is working on a shared spreadsheet generated by a spreadsheet application program 106, which is an example of a productivity application program. The user computer 102 is configured to track each of these user-specific interactions by collecting computing information that is specific to the human subject 100. This computing information is a form of posture assessment signals that are collected for purposes of assessing the human subject's posture.

Furthermore, while the human subject 100 is interacting with the computer 102, various posture assessment sensors acquire posture assessment signals associated with the human subject 100. As one example, a camera 108 captures images of the human subject 100. As another example, a microphone 110 acquires an audio signal corresponding to the human subject's voice.

The user computer 102 is configured to collect the various posture assessment signals for the human subject 100 in strict accordance with user-authorized privacy settings. When applicable, the computing information representing the various posture assessment signals may include a range of different types of information, which may be anonymized or pseudo-anonymized in accordance with user-authorized privacy settings. Such information may include raw data, parameters derived from the raw data, and/or user-state metrics that are derived from the parameters/raw data.

Whenever user information is collected for any purpose, the user information is collected with the utmost respect for user privacy (e.g., user information is only collected after the user owning the information provides affirmative consent). Whenever information is stored, accessed, and/or processed, the information is handled in accordance with privacy and/or security standards to which the user has opted in. Prior to user information being collected, users may designate how the information is to be used and/or stored, and user information may only be used for the specific, objective-driven purposes for which the user has opted in. Users may opt-in and/or opt-out of information collection at any time. After information has been collected, users may issue a command to delete the information, and/or restrict access to the information. All potentially sensitive information optionally may be encrypted and/or, when feasible anonymized or pseudo-anonymized, to further protect user privacy. Users may optionally designate portions of data, metadata, or statistics/results of processing data for release to specific, user-selected other parties, e.g., for further processing. Information that is private and/or confidential may be kept completely private, e.g., only decrypted temporarily for processing, or only decrypted for processing on a user device and otherwise stored in encrypted form. Users may hold and control encryption keys for the encrypted information. Alternately or additionally, users may designate a trusted third party to hold and control encryption keys for the encrypted information, e.g., so as to provide access to the information to the user according to a suitable authentication protocol.

Such tracking of posture assessment signals can be performed through the application programs themselves, an operating system, and/or other activity tracking services of the computer 102.

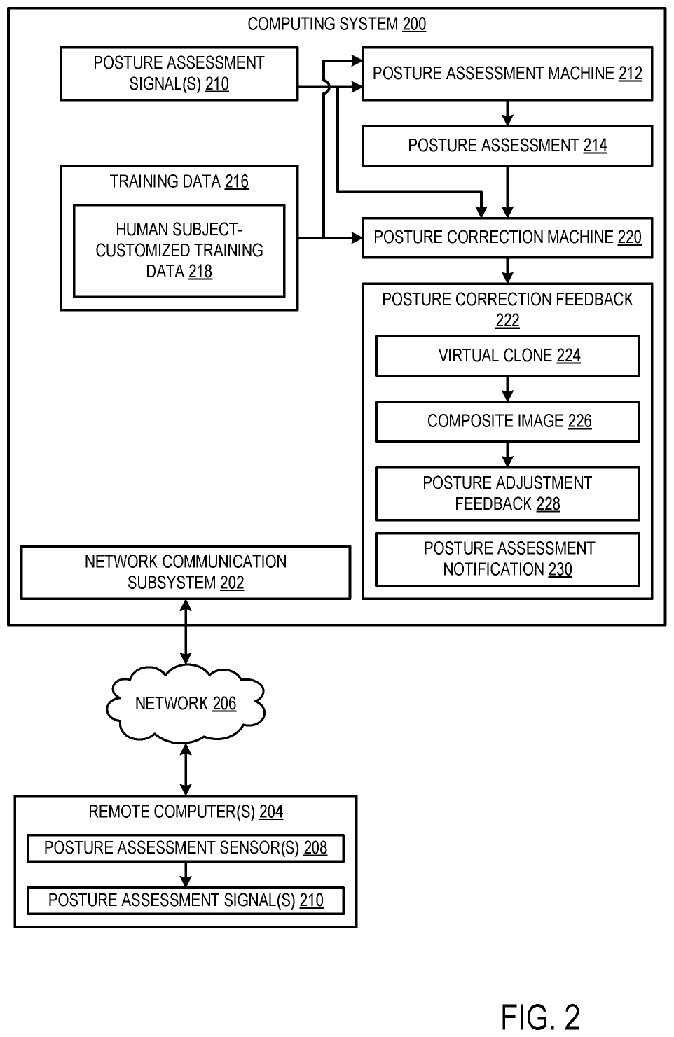

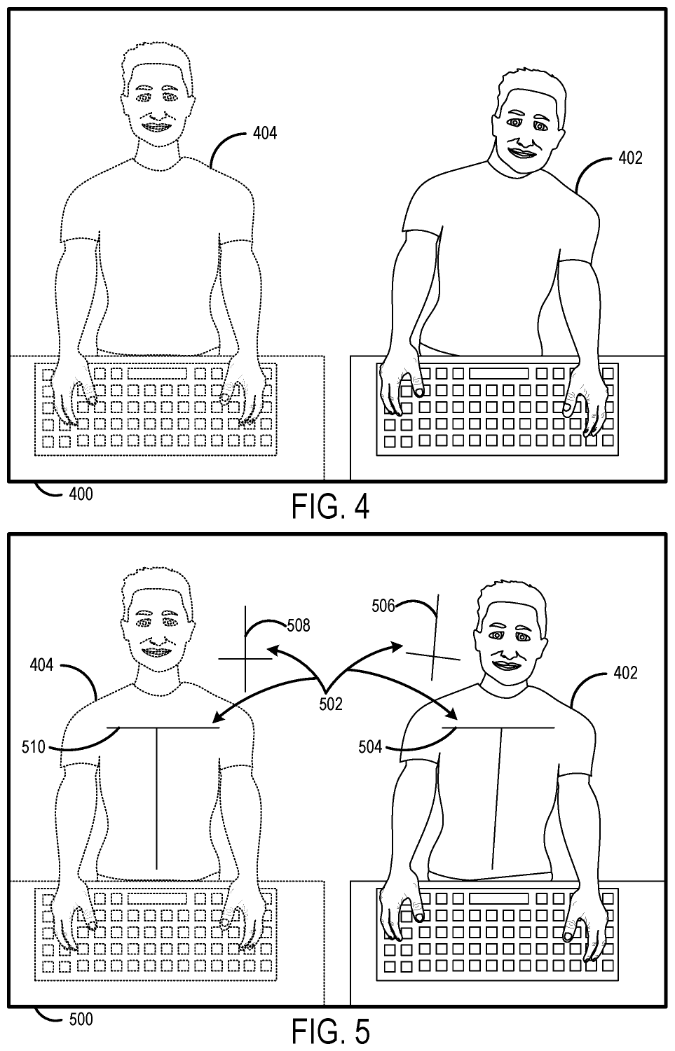

The computer 102 is configured to send the posture assessment signals to a computing system 200 (shown in FIG. 2). The computing system 200 is configured to output an assessment of the human subject's posture based at least on the posture assessment signals. Further, the computing system 200 is configured to output posture correction feedback that the human subject 100 can use to improve their posture based at least on the posture assessment signals. In some examples, the posture correction feedback is instantaneous in the sense that the posture correction feedback is based on a snapshot assessment of the human subject's current posture. In other examples, the posture correction feedback progressively tracks how the human subject's posture changes over a posture tracking duration. Various examples of posture correction feedback will be discussed in further detail below with reference to FIGS. 4-10. The computing system 200 sends the assessment of the human subject's posture and the posture correction feedback to the computer 102. The computer 102 visually presents the posture assessment and the posture correction feedback 112 to the human subject 100, such that the human subject is made aware of their posture and can use the feedback to improve their posture. Such improved posture bestows numerous benefits upon the human subject including improved health and wellbeing, increased likelihood of user interaction, and increased productivity.

In some implementations, the computer 102 is configured to provide posture assessment and/or posture correction feedback functionality without the aid of the computing system 200.

The concepts related to computer-based posture assessment and feedback functionality discussed herein are broadly applicable to any suitable type of computer or computing system including a laptop computing device, a mobile computing device (e.g., smartphone), a wearable computing device, a mixed/augmented/virtual reality computing device, or another type of user computer.

FIG. 2 shows the computing system 200 that is configured to assess a human subject's posture and provide posture correction feedback that the human subject can use to improve their posture.

In some implementations, the computing system 200 operates as a cloud service. In some such implementations, the computing system 200 includes a plurality of different computing devices that perform different operations to collectively provide posture assessment and/or posture correction functionality.

The computing system 200 includes a network communication subsystem 202 configured to communicate with one or more remote computers 204 via a computer network 206. In some examples, the remote computer(s) 204 can be associated with a human subject. For example, the remote computer(s) 204 may represent the computer 102 associated with the human subject 100 shown in FIG. 1. In some examples, the remote computer(s) 204 may represent a plurality of computer associated with a human subject, such as a work computer, a home computer, a smartphone, a laptop, a tablet, a HMD device, a game console, or another computer associated with the human subject.

In some examples, the remote computer(s) 204 can be associated with different users, and the computing system 200 may provide posture assessment and correction functionality for a plurality of different human subjects. The computing system 200 may provide posture assessment and/or posture correction functionality for any suitable number of different human subjects associated with any suitable number of remote computers via the network communication subsystem.

The remote computer(s) 204 include one or more posture assessment sensors 208 that acquire one or more posture assessment signals 210 for a human subject. The computing system 200 receives, via the network communication subsystem 202, the posture assessment signals 210 for the human subject from the remote computer(s) 204. In some examples, the computing system 200 may receive posture assessment signals for the human subject from a plurality of different remote computers 204. In one example, the posture assessment signals 210 may be received from a plurality of cloud service computers that collect/track user-specific computing information (e.g., user-images, user-audio, user-state metrics) based at least on user interactions with one or more computers.

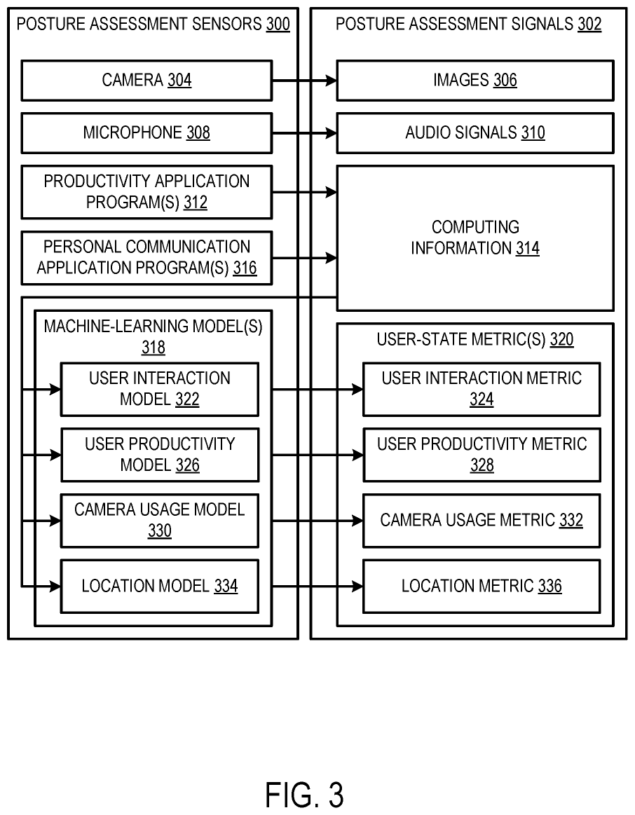

FIG. 3 shows example posture assessment sensors 300 that output posture assessment signals 302 for assessing a human subject's posture. For example, the posture assessment sensors 300 may be representative of the posture assessment sensor(s) 208 shown in FIG. 2. Further, the posture assessment signals 302 may be representative of the posture assessment signal(s) 210 shown in FIG. 2.

As used herein, a “posture assessment sensor” is any suitable source of a posture assessment signal that informs an assessment of the human subject's posture. In some examples, a posture assessment sensor may be a hardware component. In other examples, a posture assessment sensor may be a software component. Further, as used herein, a “posture assessment signal” is any suitable piece of information or data that informs an assessment of a human subject's posture.

In some examples, the posture assessment sensors 300 include a digital camera 304 that captures digital images 306 of a human subject. The camera 304 may capture any suitable type of images including, but not limited to, monochrome images, color images, hyperspectral images, depth images, thermal images, and/or other types of images. In some examples, the camera 304 captures a sequence of images of a human subject—i.e., a video of the human subject. In some examples, the camera 304 may be a peripheral device, such as a peripheral “web camera.” In other examples, the camera 304 may be integral to a computer, such as a camera in a laptop, smartphone, or a head-mounted display (HMD) device. In some examples, the camera 304 captures the image(s) 306 of the human subject through a dedicated posture assessment application program. In other examples, the camera 304 captures the image(s) 306 of the human subject opportunistically when a different application program uses the camera 304, such as when the camera 304 is used to capture images of the human subject for a video conference call carried out by a video conference application program.

The image(s) 306 of the human subject provide the most direct and accurate information about the human subject's posture relative to other posture assessment signals. In some examples, a single image can be captured for assessment of a human subject's posture. In other examples, a series of images can be captured over a continuous duration for assessment of a human subject's posture (e.g., 1 second, 10 seconds, 30 seconds, 1 minute, 5 minutes, or longer). In still other examples, a plurality of images of the human subject can be captured at different intervals over a posture tracking duration for posture assessment and posture tracking (e.g., different intervals across hours, days, week, month, years, or longer).

In some examples, the posture assessment sensors 300 include a microphone 308 that acquires posture assessment signals in the form of audio signals 310 corresponding to a human subject's voice. For example, the audio signals 310 corresponding to the human subject's voice may be acquired while the human subject is interacting with a computer, such as during an audio call or a video conference. The audio signals 310 corresponding to the human subject's voice can be analyzed to link characteristics of the human subject's voice to the human subject's posture. For example, the human subject's posture can be assessed based on a volume and/or a tone of the human subject's voice. In some examples, a change in characteristics of the human subject's voice can indicate a change in the human subject's posture. Any suitable characteristics of the human subject's voice can be analyzed to assess the human subject's posture.

In some examples, the posture assessment sensors 300 include one or more productivity application programs 312 that generate posture assessment signals in the form of computing information 314 corresponding to user-interactions of a human subject. The productivity application program(s) 312 may include any suitable type of application program that promotes user productivity. Non-limiting examples of productivity application programs 312 include word processing application programs, spreadsheet application programs, slide deck presentation application programs, note taking application programs, drawing/diagraming application programs, calendar application programs, and browser application programs. The computing information 314 generated by the productivity application program(s) 312 indicate various aspects of user interactions that can inform an assessment of a human subject's posture. The computing information 314 generated by the productivity application program(s) 312 may take any suitable form. Non-limiting examples of such computing information may include the frequency at which different productivity application programs are used by the user, the computer/location from which the user uses different productivity application programs, other users that the user interacts with while using different productivity application programs, and language (written/typed or spoken) used in different productivity application programs.

In some examples, the posture assessment sensors 300 include one or more personal communication application programs 316 that generate computing information 314. The personal communication application program(s) 316 may include any suitable type of application program that promotes user communication with other users. Non-limiting examples of personal communication application programs 316 include email application programs, messaging application programs, audio application programs, video application programs, audio/video conferencing application programs, and social network application programs. The computing information 314 generated by the personal communication application program(s) 316 may indicate various aspects of user interactions that can inform an assessment of a human subject's posture. The computing information 314 generated by the personal communication application program(s) 316 may take any suitable form. Non-limiting examples of such computing information may include email messages, text messages, comments posted by a user in a document or file, audio transcripts, user audio segments, and user video segments, the frequency at which different personal communication application programs are used by the user, the computer/location from which the user uses different personal communication application programs, other users that the user interacts with while using different personal communication application programs, language (written/typed or spoken) used in different personal communication application programs.

The computing information 314 may be aggregated for a human subject over multiple different virtual interactions with different application programs and/or other users via the productivity application program(s) 312, the personal communication application program(s) 316, other application programs, an operating system, and/or computing services. Further, in some examples, application programs executing on a computer may be configured to obtain user-specific computing information in other manners, such as explicitly requesting the computing information 314 from the user and/or inferring the computing information 314 based at least on user actions. The computing information 314 may be obtained for a user in any suitable manner.

In some examples, the posture assessment sensors 300 include one or more machine-learning models 318 that output posture assessment signals in the form of one or more user-state metrics 320. The machine-learning model(s) 318 may be previously-trained to quantify factors that contribute to an assessment of a human subject's posture based at least on the computing information 314 acquired for the human subject in the form of the user-state metric(s) 320. The user-state metric(s) 320 indicate higher-level information that is distilled down from raw data and processed by the machine-learning model(s) 318.

In one example, the machine learning model(s) 318 include a user interaction model 322 previously-trained to output a user interaction metric 324 indicating a level of user interaction of the human subject based at least on the computing information 314 for the human subject. For example, the user interaction metric 324 may track a frequency of communications (e.g., emails, messages, comments) from the human subject to other users, a frequency that the human subject attends and/or initiates scheduled interactions (e.g., via audio calls, video conferences), a frequency that the human subject is intentionally invited by other users to interact, and/or another suitable quantifier of a level of user interaction. The user interaction model 322 may determine the level of user interaction of the human subject in any suitable manner. The level of interaction quantified by the user interaction metric 324 provides insight into a human subject's wellbeing and by association their posture. For example, if a level of human interaction of a human subject reduces in a statistically significant manner over a designated timeframe, then such behavior may indicate that the human subject's wellbeing is decreasing and their posture is getting worse. On the other hand, a human subject having a higher level of interaction is more likely to have good posture.

In another example, the machine learning model(s) 318 include a user productivity model 326 previously-trained to output a user productivity metric 328 indicating a level of user productivity based at least on the computing information 314. A user's level of productivity may be determined based at least on a variety of factors including, but not limited to, a user input speed, a task completion time, a time taken for a user to take action responsive to a notification and/or to return to a previous task after taking action responsive to a notification. The user productivity model 326 may determine the level of user productivity in any suitable manner. The level of productivity quantified by the user productivity metric 328 provides insight into a human subject's wellbeing and by association their posture. For example, if a level of productivity of a human subject reduces in a statistically significant manner over a designated timeframe, then such behavior may indicate that the human subject's wellbeing is decreasing and their posture is getting worse. On the other hand, a human subject having a higher level of productivity is more likely to have good posture.

In another example, the machine learning model(s) 318 include a camera usage model 330 previously-trained to output a camera usage metric 332 indicating a level of camera usage during user interactions facilitated by the personal communication application program(s) 316. The camera usage model 330 may receive computing information 314 indicating each time a user's camera is turned on during a user interaction. Such camera usage may be reported by the personal communication application program(s) 316. In one example, the camera usage metric 332 may be represented as a scalar between 0-100, where 0 corresponds to a user not using the camera at all and 100 corresponding to a user using the camera during every user interaction. The camera usage model 330 may determine the level of camera usage in any suitable manner. The level of camera usage quantified by the camera usage metric 332 provides insight into a human subject's wellbeing and by association their posture. For example, if a level of camera usage of a human subject reduces in a statistically significant manner over a designated timeframe, then such behavior may indicate that the human subject's wellbeing is decreasing and their posture is getting worse. On the other hand, a human subject having a higher level of camera usage is more likely to have good posture.

In another example, the machine learning model(s) 318 include a location model 334 previously-trained to output a location metric 336 indicating a level to which a human subject's location changes on an interaction-to-interaction basis when interacting with the productivity application program(s) 312, the personal communication application program(s) 316, and/or any other application programs. In one example, the location model 334 may be configured to track a human subject's location based at least on logging IP addresses of computers when the human subject user interacts with different application programs. The location model 334 may be configured to track the human subject's location in any suitable manner to generate the location metric 336. Further, the location model 334 may determine the level to which the human subject's location changes on an interaction-to-interaction basis in any suitable manner. The level to which a human subject's location changes on an interaction-to-interaction basis provides insight into the human subject's wellbeing and by association their posture. For example, if the human subject goes from working from different public locations (e.g., a restaurant or coffee shop) on a regular basis to working from the same private location (e.g., the human subject's mother's basement) during a designated timeframe, then such a change in behavior may indicate that the human subject's wellbeing is decreasing and the posture is getting worse. On the other hand, a human subject that changes locations of interaction more often is more likely to have good posture.

Any suitable number of different machine-learning models 318 that output any suitable user-state metric can be used to generate posture assessment signals 302 to assess a human subject's posture. In some examples, one or more of the machine-learning models 318 may be previously-trained neural networks.

While machine-learning models can advantageously diagnose and summarize complicated user behavior patterns and associate such behavior patterns with a human subject's posture, in some implementations hard-coded heuristics or other assessment logic may be used in addition to or instead of machine-learning models to assess a human subject's posture.

In some implementations, one or more of the machine-learning models 318 is executed by the computing system 200 (shown in FIG. 2). In some implementations, one or more of the machine-learning models 318 is executed by a user computer, such as the computer 102 (shown in FIG. 1). Such processing performed using the computing resources of the computer 102 reduces an amount of information/data that is sent to the computing system 200 relative to a configuration where a centralized computing system processes all the raw data unassisted.

Additionally or alternatively, in some implementations, one or more of the machine-learning models 318 is executed by one or more other remote computers 204 (shown in FIG. 2), such as different computers dedicated to generating different user-metrics, in a cloud service, for example. Such processing performed using the computing resources of the other remote computers reduces a processing burden of the computing system 200 (shown in FIG. 2) relative to a configuration where a centralized computing system processes all the raw data unassisted.

These features provide the technical benefits of providing increased performance for the computing system 200 to generate posture assessments and reduced data transmission that equates to reduced power consumption and increases the amount of communication bandwidth available for other communications. In some implementations, raw data may be sent from a user computer to a central computing system for remote processing; and in some implementations a combination of local and remote processing may be employed. In still other implementations, processing may be performed locally on a single computer.

Returning to FIG. 2, the computing system 200 includes a posture assessment machine 212 that receives the one or more posture assessment signals 210 from the one or more posture assessment sensors 208. The posture assessment machine 212 outputs a posture assessment 214 of a human subject's posture based at least on the one or more posture assessment signals 210.

In some examples where the posture assessment sensor(s) 208 include a camera, the posture assessment machine 212 receives a plurality of images of the human subject captured by the camera and outputs the posture assessment 214 based at least on the plurality of images. In some examples where the posture assessment sensor(s) 208 include a microphone, the posture assessment machine 212 receives an audio signal corresponding to the human subject's voice acquired by the microphone, and outputs the posture assessment 214 based at least on the audio signal. In some examples, the posture assessment signal(s) 210 include one or more user-state metrics 320 output from one or more trained machine-learning models 318 shown in FIG. 3, and the posture assessment machine 212 outputs the posture assessment 214 based at least on the one or more user-state metrics 320. Example user-state metrics that can be used generate the posture assessment 214 include the user interaction metric 324, the user productivity metric 328, the camera usage metric 332, and the location metric 336. The posture assessment machine 212 may be configured to generate the posture assessment 214 based on any suitable user-state metric.

In some examples, the posture assessment machine 212 is configured to generate the posture assessment 214 based on a plurality of posture assessment signals 210. In some examples, the plurality of posture assessment signals 210 may be arranged in a multi-dimensional vector data structure, and the posture assessment machine 212 outputs the posture assessment 214 based at least on the multi-dimensional vector data structure. In one example, a multi-dimensional vector data structure includes images 306, audio signals 310, and a plurality of user-state metrics 320.

In some examples, the posture assessment machine 212 is configured to generate the posture assessment 214 based at least on different posture assessment signals when those posture assessment signals are available. For example, when the posture assessment machine 212 receives images of the human subject, the posture assessment machine 212 generates the posture assessment 214 based at least on the images of the human subject. In another example, when the posture assessment machine 212 receives images of the human subject and an audio signal of the human subject's voice, the posture assessment machine 212 generates the posture assessment 214 based at least on the images of the human subject and the audio signal of the human subject's voice. In yet another example, when the audio signal of the human subject's voice is available and images of the human subject are not available, the posture assessment machine 212 generates the posture assessment 214 based at least on the audio segment of the human subject's voice. Such a posture assessment, in some cases, may be less accurate than a posture assessment generated based at least on both images and an audio signal, but the strictly audio-based posture assessment still provides some degree of posture assessment accuracy. By assessing a human subject's posture based on different posture assessment signals when they are available, the posture assessment machine 212 can output a robust assessment of a human subject's posture under varying operating conditions and device capabilities.

The posture assessment machine 212 may be configured to generate the human subject's posture assessment 214 in any suitable manner. In one example, the posture assessment machine 212 includes a previously-trained machine-learning model, such as a neural network. In particular, the machine-learning model may be previously-trained to receive the posture assessment signal(s) 210 as input and output the human subject's posture assessment 214 based at least on the posture assessment signal(s) 210. The machine-learning model may be trained using training data 216 that includes various posture assessment signals. For example, such posture assessment signals may include images of human subject assuming different postures, audio signals of human subject's voices while assuming different postures, and/or user-state metrics of different human subjects having different postures.

The human subject's posture assessment 214 may take any suitable form. In some examples, the posture assessment 214 may include a descriptive label, such as “poor”, “adequate”, or “good”. In some examples, the posture assessment 214 may include a number (e.g., an integer/scalar). In other examples, the posture assessment 214 may include a multi-dimensional vector (e.g., represented as a vector with a plurality of coefficients relating to different aspects of a human subject's posture—e.g., neck position, back position, shoulder position, arm position).

In some implementations, the posture assessment machine 212 is configured to progressively update the human subject's posture assessment 214 over a posture tracking duration based at least on the posture assessment signal(s) 210. In other words, as the posture assessment signals 210 are updated over time, the posture assessment machine 212 updates the human subject's posture assessment 214 based at least on the updated posture assessment signals 210. The human subject's posture assessment 214 may be progressively updated over time in order to observe and track changes in the human subject's posture. The posture assessment machine 212 may update the human subject's posture assessment 214 according to any suitable frequency and/or any suitable posture tracking duration that allows for such observation and tracking of changes in the human subject's posture.

In some examples, the posture assessment 214 may be represented over time as a function of a variable, and the change in posture may be represented by the first derivative of this function and/or the net change in this value over a certain period of time. As another example, in the case of the posture assessment 214 being represented by a multi-dimensional vector, a change in posture may be calculated as a geometric distance between such vectors at different times. These are just examples, and other mechanisms for representing the assessment of human subject's posture and/or calculating a rate of change of the assessment of the human subject's posture may be used.

In some implementations, the posture assessment machine 212 is configured to be updated/re-trained to customize the posture assessment 214 based on feedback from the human subject. In one example, the training data 216 includes a plurality of training images of training clones of the human subject having different “correct” postures. The training clones are visually presented to the human subject, and the human subject selects a “best-fit” clone from the plurality of training clones that the human subject deems to be the most accurate representation of the correct posture. Further, the posture assessment machine 212 is updated/retrained to customize the posture assessment 214 based at least on the best-fit clone selected by the human subject. In this example, the human subject selected best-fit clone represents human subject-customized training data 218. The posture assessment machine 212 can be updated/re-trained to customize the posture assessment 214 based on any suitable human subject-customized training data 218. Such a feature provides the technical benefit of improving accuracy of the posture correction machine 212 to assess a human subject's posture on an individual human subject basis.

The computing system 200 includes a posture correction machine 220 configured to receive one or more posture assessment signals 210 and the posture assessment 214 of the human subject's posture. The posture correction machine 220 is configured to output posture correction feedback 222 based at least on the one or more posture assessment signals 210 and/or the posture assessment 214 of the human subject's posture.

The posture correction machine 220 may be configured to generate the posture correction feedback 222 in any suitable manner. In one example, the posture correction machine 220 includes a previously-trained machine-learning model, such as a neural network. In particular, the machine-learning model may be previously-trained to receive the posture assessment signal(s) 210 and the posture assessment 214 as input and output the posture correction feedback 222 based at least on the posture assessment signal(s) 210 and the posture assessment 214.

The posture correction feedback 222 may take any suitable form. In some examples, the posture correction feedback 222 is instantaneous in the sense that the posture correction feedback 222 is based on a snapshot assessment of the human subject's current posture.

In some examples, the posture correction machine 220 is configured to receive one or more images 306 of the human subject (shown in FIG. 3) and generate a virtual clone 224 of the human subject based at least on the images 306 of the human subject. The virtual clone 224 has an improved posture relative to the human subject's posture as assessed by the posture assessment machine 212. The virtual clone 224 is a virtual replica of the human subject created from the images 306 of the human subject by the posture correction machine 220 using artificial intelligence. In some examples, the virtual clone 224 may be a photo-realistic representation of the human subject. In other examples, the virtual clone 224 may be more stylized. For example, the virtual clone 224 may include stylized features that emphasize which body parts of the human subject need adjustment to improve the human subject's posture.

In one example, the posture correction machine 220 includes one or more generative adversarial networks (GANs) that are trained to output the virtual clone 224 using training data 216 including sets of training images of the human subject with different postures (e.g., some images with correct posture and some images with poor posture). That way, given any new image x_i of a human subject with a given posture as input, the trained GANs can predict the correct posture Gt(x_i) of the human subject while still preserving personalized features of the human subject in the virtual clone 224.

In some implementations, the posture correction machine 220 is configured to be updated/re-trained to customize the posture correction feedback 222 based on feedback from the human subject. In one example, a plurality of training clones of the human subject having different postures is visually presented to the human subject. The human subject selects, via user input, a selection of a best-fit clone of the plurality of training clones that the human subject deems to have the most accurate representation of the proper posture. The posture correction machine 220 is configured to be updated/re-trained to customize a posture of the virtual clone 224 based at least on the best-fit clone selected by the human subject via user input. This feature provides the technical benefit of increasing posture correction accuracy on an individual human subject basis that improves human computer interaction.

The posture correction machine 220 is configured to generate a composite image 226 including the virtual clone 224 admixed with an image of the human subject. The composite image 226 provides a visual representation of the human subject's current posture as compared to the improved posture of the virtual clone 224 that the human subject can use as a reference to improve their actual posture. In some examples, the posture correction machine 220 is configured to admix posture adjustment feedback 228 into the composite image 226. The posture adjustment feedback 228 visually indicates whether the human subject's posture approaches the improved posture of the virtual clone 224. The posture adjustment feedback 228 may take any suitable form.

The posture correction machine 220 is configured to send the composite image 226 to a remote computer 204 associated with the human subject (e.g., the computer 102 shown in FIG. 1), and the remote computer 204 is configured to visually present the composite image 226 to the human subject for posture correction. In some implementations, the remote computer 204 visually presents the composite image 226 in a dedicated posture correction application program. In some implementations, the remote computer 204 visually presents the composite image 226 as a productivity feature integrated into a different application program, such as the productivity application program 312 and/or the personal communication application program 316. In some implementations, the remote computer 204 visually presents the composite image 226 based at least on a user request to manually check the posture of the human subject. In some implementations, the remote computer 204 automatically visually presents the composite image 226 based at least on the posture assessment 214 of the human subject falling below a posture assessment threshold. For example, the remote computer 204 may automatically visually present the composite image 226 based on the posture assessment 214 indicating that the human subject has poor posture.

FIG. 4 shows an example composite image 400 including an image of a human subject 402 admixed with a virtual clone 404. For example, the composite image 400 may represent the composite image 226 including the virtual clone 224 shown in FIG. 2. The virtual clone 404 has an appearance that corresponds to the appearance of the human subject 402. In some examples, the virtual clone 404 is a photo-realistic representation of the human subject 402 generated from images of the human subject 402. In other examples, the virtual clone 404 is a stylized version of the human subject 402. The virtual clone 404 has an improved posture relative to the posture of the human subject 402. In the illustrated example, the human subject 402 is leaning to one side and hunched over with a bent neck. On the other hand, the virtual clone 404 is standing up straight with square shoulders. Further, the virtual clone's head is vertically aligned with the spine and the neck is extended. The composite image 400 provides a visual reference that the human subject 402 can use to adjust their posture to approach the improved posture of the virtual clone 404.

FIG. 5 shows an example composite image 500 including posture adjustment feedback 502. The composite image 500 is generated subsequent to the composite image 400 shown in FIG. 4 when the human subject 402 has adjusted their posture. The posture adjustment feedback 502 indicates whether the posture of the human subject 402 approaches the improved posture of the virtual clone 404. In the illustrated implementation, the posture adjustment feedback 502 includes different sets of axes corresponding to different body parts of the human subject 402. A first set of axes 504 is associated with the human subject's torso and indicates whether the human subject's spine is straight, and the shoulders are square with the spine. A second set of axes 506 is associated with the human subject's neck and head and indicates whether the human subject's neck is straight, and the head is square with the neck. Further, the virtual clone 404 is annotated with corresponding sets of axes 508 and 510 that are straight and perpendicular indicating that the virtual clone's spine is straight, and shoulders are square with the spine, and the virtual clone's neck is straight, and the head is square with the neck. The different sets of axes are one example of posture adjustment feedback. The posture adjustment feedback may take any suitable form that indicates whether the posture of the human subject approaches the improved posture of the virtual clone.

Note that the composite images 400 and 500 may be generated at any suitable frequency/frame rate. In some examples, composite images may be generated in substantially real-time, such that a composite video of the human subject and the virtual clone can be visually presented to the human subject for posture correction. In such examples, the virtual clone may move as the human subject moves while maintaining the correct posture, such that the virtual clone can mimic the behavior of the human subject in a life-like fashion.

Presenting the virtual clone of the human subject in the composite image provides a customized visual representation of the human subject that enables the human subject to accurately adjust their own posture to approach the correct posture of the virtual clone. Presenting the virtual clone as posture correction feedback provides the technical benefit of improved human computer interaction through improving the human subject's posture while the human subject interacts with a computer.

Returning to FIG. 2, in some implementations, the posture assessment machine 212 is configured to receive a plurality of posture assessment signals 210 from the posture assessment sensor(s) 208 over a posture tracking duration, and progressively update the posture assessment 214 over the posture tracking duration based at least on the plurality of posture assessment signals. The posture tracking duration may include any suitable length of time (e.g., hours, days, week, month, years, or longer). The posture assessment machine 212 may progressively update the posture assessment 214 according to any suitable update rate (e.g., a rate corresponding to a frame rate of a camera or a time interval, such as a second, a minute, or a longer time interval).

Further, the posture correction machine 220 is configured to generate a posture assessment notification 230 based at least on the progressively updated posture assessments 214 of the human subject's posture over the posture tracking duration. The posture assessment notification 230 visually summarizes how the human subject's posture changed over the posture tracking duration. The posture assessment notification 230 can visually summarize the changes in the human subject's posture in any suitable manner.

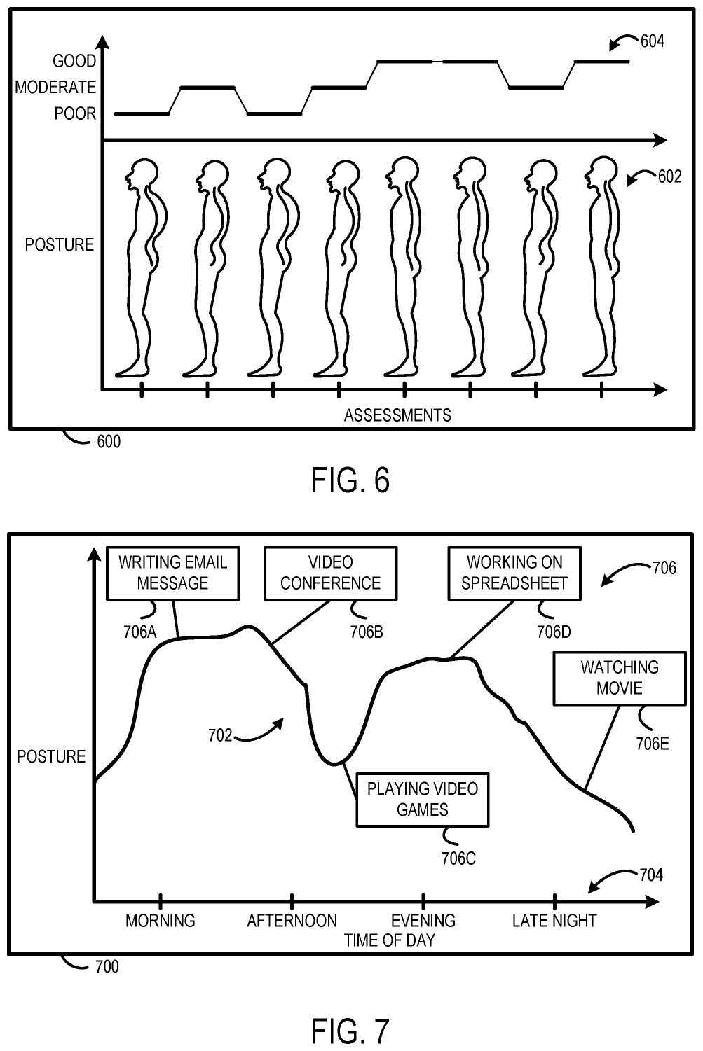

FIGS. 6-10 show different example posture assessment notifications. In examples where the posture assessment machine 212 progressively updates the posture assessment 214 based on a plurality of images of the human subject captured throughout the posture tracking duration, the posture assessment notification 230 may be derived from the plurality of images. FIG. 6 shows an example posture assessment notification 600 including a plurality of images 602 of a human subject captured over a posture tracking duration. For example, the posture assessment notification 600 may be representative of the posture assessment notification 230 shown in FIG. 2. Additionally, the posture assessment notification 600 includes a plurality of posture assessments 604 corresponding to the plurality of images 602 of the human subject. The plurality of posture assessments 604 allows the human subject to evaluate each of the different images 602. Moreover, the plurality of images 602 and corresponding posture assessments 604 provides visual evidence of how the human subject's posture changes throughout the posture tracking duration. The posture assessment notification 600 is provided as a non-limiting example of how changes in the human subject's posture over the posture tracking duration can be visually summarized.

FIG. 7 shows another example posture assessment notification 700. For example, the posture assessment notification 700 may be representative of the posture assessment notification 230 shown in FIG. 2. The posture assessment notification 700 includes a visual representation in the form of a graph 702 of a human subject's posture during different time intervals 704 during the posture tracking duration. The graph 702 may be derived from a plurality of posture assessments. In some examples, the plurality of posture assessments may be generated based on a plurality of images of the human subject captured at the different time intervals during the posture tracking duration. In the illustrated example, the graph 702 is continuous. In other examples, the graph may represent discrete assessments of the human subject's posture. In the illustrated example, the time intervals 704 correspond to different parts of a day (e.g., morning, afternoon, evening, late night). The graph 702 enables the human subject to identify time intervals in which the human subject has poor posture, so that the human subject can be mindful of such time intervals and work toward improving their posture during those same time intervals in the future. For example, the graph 702 indicates that the human subject had poor posture in the afternoon and late at night, so in the future the human subject can be aware and try to improve their posture during those time intervals.

Additionally, the posture assessment notification 700 includes context tags 706 (e.g., 706A, 706B, 706D, 706E) indicating different activities the human subject was involved in during the different time intervals 704. For example, the context tags 706 can be generated from the computing information 314 (shown in FIG. 3) generated from the human subject interacting with a computer. The context tags 706 help the human subject identify activities that may lead to the human subject having poor posture. The context tags 706 enable the human subject to identify activities in which the human subject has poor posture, so that the human subject can be mindful of such activities and work toward improving their posture while participating in those activities in the future. For example, the graph 702 and the context tag 706C indicate that the human subject had poor posture while playing video games. Further, the graph 702 and the context tag 706E indicate that the human subject had poor posture while watching a movie. The human subject is made aware of their poor posture while participating in these activities based on posture assessment notification 700, and the human subject can try to improve their posture while playing video games and watching movies in the future.

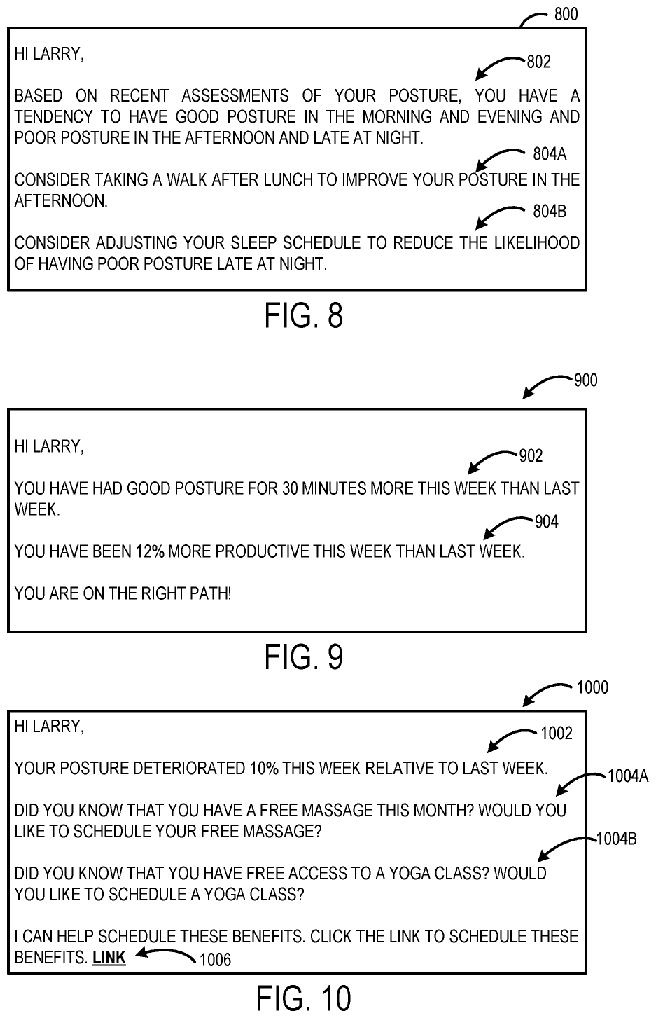

FIG. 8 shows another example posture assessment notification 800. For example, the posture assessment notification 800 may be representative of the posture assessment notification 230 shown in FIG. 2. The posture assessment notification 800 includes a visual representation in the form of a text-based message 802 that indicates how a human subject's posture changes at different intervals. In the illustrated example, the message 802 indicates that the human subject has good posture in the morning and evening and poor posture in the afternoon and late at night. The posture assessment notification 800 further includes a plurality of recommendations 804 (e.g., 804A, 804B) that the human subject can enact to improve their posture. As one example, the recommendation 804A suggests that the human subject take a walk after lunch to improve their posture in the afternoon. As another example, the recommendation 804B suggests that the human subject adjust their sleep schedule to reduce the likelihood of having poor posture late at night. The posture assessment notification 800 may include any suitable recommendations to improve a human subject's posture.

FIG. 9 shows another example posture assessment notification 900. For example, the posture assessment notification 900 may be representative of the posture assessment notification 230 shown in FIG. 2. The posture assessment notification 900 includes a visual representation in the form of a text-based message 902 that indicates that a human subject had good posture for 30 more minutes this week than last week. In this example, the message 902 provides a comparison of posture assessments from different intervals (e.g., week-to-week) during a posture tracking duration to inform the human subject how the human subject's posture has changed in a positive manner.

Additionally, the posture assessment notification 900 includes a benefits notification 904 indicating posture improving benefits realized by the human subject based on the week-over-week improvement of the human subject's posture. In the illustrated, the benefits notification 904 indicates that the human subject was 12% more productive this week than last week. For example, the human subject's productivity can be tracked via the user productivity metric 328 (shown in FIG. 3). In this example, the benefits notification 904 shows the human subject how improvements in the human subject's posture are linked to improvements in the human subject's productivity. The benefits notification 904 may indicate any suitable benefit of having improved posture that can be tracked by a computer based on user interaction of the human subject with the computer.

FIG. 10 shows another example posture assessment notification 1000. For example, the posture assessment notification 1000 may be representative of the posture assessment notification 230 shown in FIG. 2. The posture assessment notification 1000 includes a visual representation in the form of a text-based message 1002 that indicates that a human subject's posture has deteriorated 10% this week relative to last week. In this example, the message 1002 provides a comparison of posture assessments from different intervals (e.g., week-to-week) during a posture tracking duration to inform the human subject how the human subject's posture has changed in a negative manner.

Additionally, the posture assessment notification 1000 includes a plurality of benefits notifications 1004 (e.g., 1004A, 1004B) indicating posture improving benefits that are currently available for the human subject to improve their posture. The benefits notification 1004A indicates that the human subject has a benefit for a free message (e.g., as part of an employee benefits package). The benefits notification 1004B indicates that the human subject has free access to a yoga class. The posture assessment notification 1000 includes a scheduling prompt 1006 that is selectable via user input to automatically schedule times for the human subject to use the free benefits. The plurality of benefits notifications 1004 present proactive steps that the human subject can take to improve the human subject's posture.

A posture assessment notification can visually summarize changes in a human subject's posture in any suitable manner. Further, a posture assessment notification can provide any suitable benefit notification that indicate benefits that result from having good posture and recommendations of benefits (or activities) that the human subject can participate in to improve their posture.

In some implementations, a posture assessment notification can be visually presented to provide an instantaneous indication of a human subject's posture instead of tracking change of a human subject's posture over a posture tracking duration. For example, whenever a posture assessment indicates that a human subject's posture is poor (or below a threshold level), a posture assessment notification may be visually presented to notify the human subject that their posture is poor, so that the human subject can improve their posture.

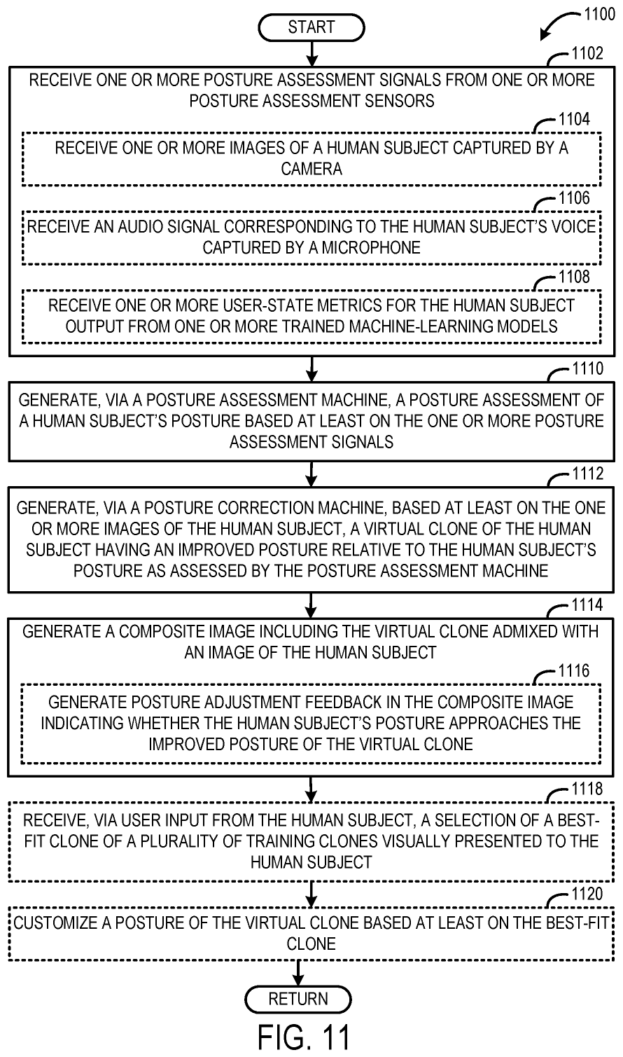

FIG. 11 shows an example computer-implemented method 1100 for assessing and correcting a human subject's posture. For example, the computer-implemented method 1100 may be performed by the computing system 200 shown in FIG. 2.

At 1102, the computer-implemented method 1100 includes receiving one or more posture assessment signals from one or more posture assessment sensors. In some implementations, at 1104, the computer-implemented method 1100 may include receiving one or more images of a human subject captured by a camera. In some implementations, at 1106, the computer-implemented method 1100 may include receiving an audio signal corresponding to the human subject's voice captured by a microphone. In some implementations, at 1108, the computer-implemented method 1100 may include receiving one or more user-state metrics for the human subject output from one or more trained machine-learning models.

At 1110, the computer-implemented method 1100 includes generating, via a posture assessment machine, a posture assessment of a human subject's posture based at least on the one or more posture assessment signals.

At 1112, the computer-implemented method 1100 includes generating, via a posture correction machine, based at least on the one or more images of the human subject, a virtual clone of the human subject having an improved posture relative to the human subject's posture as assessed by the posture assessment machine.

At 1114, the computer-implemented method 1100 includes generating, via the posture correction machine, a composite image including the virtual clone admixed with an image of the human subject. The composite image may be sent to a user computer via a computer network for visual presentation to the human subject. In some implementations, at 1116, the computer-implemented method 1100 may include generating posture adjustment feedback in the composite image. The posture adjustment feedback indicates whether the human subject's posture approaches the improved posture of the virtual clone.

In some implementations, at 1116, the computer-implemented method 1100 may include receiving, via user input from the human subject, a selection of a best-fit clone of a plurality of training clones visually presented to the human subject. For example, the plurality of training clones may be visually presented to the human subject in a training or calibration session as part of customizing the posture correction machine. In some implementations, at 1118, the computer-implemented method 1100 may include customizing, via the posture correction machine, a posture of the virtual clone based at least on the best-fit clone.

The above-described computer-implemented method may be performed to provide posture assessment and feedback for posture correction. In particular, by generating the composite image including both the human subject and the virtual clone, the human subject is provided with a visual comparison that the human subject can use to correct the human subject's posture.

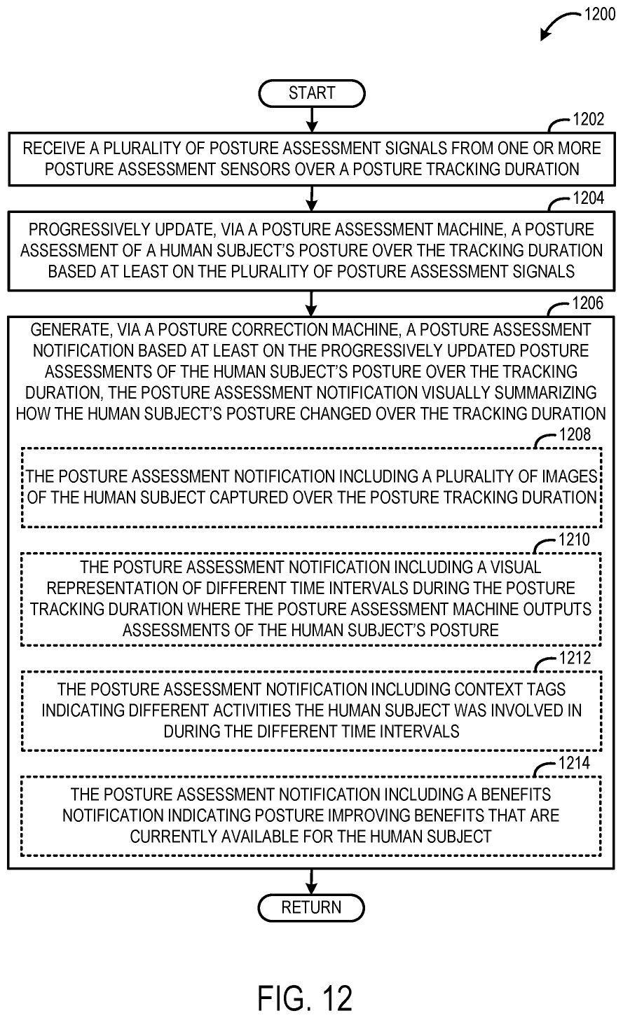

FIG. 12 shows an example computer-implemented method 1200 for progressively assessing a human subject's posture over a posture tracking duration. For example, the computer-implemented method 1100 may be performed by the computing system 200 shown in FIG. 2.

At 1202, the computer-implemented method 1200 includes receiving a plurality of posture assessment signals from one or more posture assessment sensors over a posture tracking duration.

At 1204, the computer-implemented method 1200 includes progressively updating, via a posture assessment machine, a posture assessment of a human subject's posture over the posture tracking duration based at least on the plurality of posture assessment signals.

At 1206, the computer-implemented method 1200 includes generating, via a posture correction machine, a posture assessment notification based at least on the progressively updated assessments of the human subject's posture over the posture tracking duration. The posture assessment notification visually summarizes how the human subject's posture changed over the posture tracking duration. In some implementations, at 1208, the posture assessment notification may include a plurality of images of the human subject captured over the posture tracking duration. In some implementations, at 1210, the posture assessment notification may include a visual representation of different time intervals during the posture tracking duration where the posture assessment machine outputs assessments of the human subject's posture. In some implementations, at 1212, the posture assessment notification may include context tags indicating different activities the human subject was involved in during the different time intervals. In some implementations, at 1214, the posture assessment notification may include a benefits notification indicating posture improving benefits that are currently available for the human subject.

The above-described computer-implemented method may be performed to allow a human subject to track changes in their posture over a posture tracking duration. In particular, by providing a visual summary of how the human subject's posture changes over the posture tracking duration, the human subject is able to recognize a distinct change in the human subject's posture and make adjustments as needed to improve their posture.

The above-described computer-implemented methods provide the technical benefit of improving human computer interaction by assessing and correcting a human subject's posture while the human subject interacts with a computer. Such an improved posture can positively affect the human subject's wellbeing, collaboration with other people, and overall productivity.

The methods and processes described herein may be tied to a computing system of one or more computing devices. In particular, such methods and processes may be implemented as an executable computer-application program, a network-accessible computing service, an application-programming interface (API), a library, or a combination of the above and/or other compute resources.

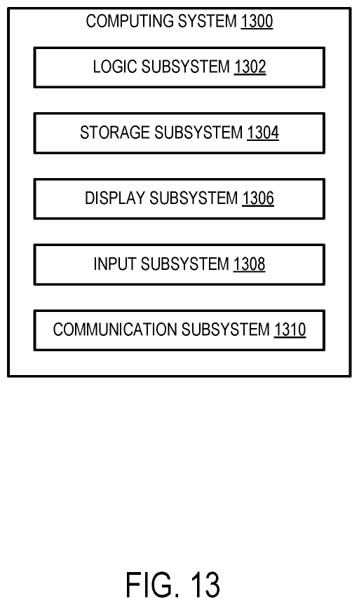

FIG. 13 schematically shows a simplified representation of a computing system 1300 configured to provide any to all of the compute functionality described herein. For example, the computing system 1300 may correspond to the user computer 102 shown in FIG. 1, the computing system 200 and the remote computer(s) 204 shown in FIG. 2. Computing system 1300 may take the form of one or more personal computers, network-accessible server computers, tablet computers, home-entertainment computers, gaming devices, mobile computing devices, mobile communication devices (e.g., smart phone), virtual/augmented/mixed reality computing devices, wearable computing devices, Internet of Things (IoT) devices, embedded computing devices, and/or other computing devices.

Computing system 1300 includes a logic subsystem 1302 and a storage subsystem 1304. Computing system 1300 may optionally include a display subsystem 1306, input subsystem 1308, communication subsystem 1310, and/or other subsystems not shown in FIG. 13.

Logic subsystem 1302 includes one or more physical devices configured to execute instructions. For example, the logic subsystem may be configured to execute instructions that are part of one or more applications, services, or other logical constructs. The logic subsystem may include one or more hardware processors configured to execute software instructions. Additionally or alternatively, the logic subsystem may include one or more hardware or firmware devices configured to execute hardware or firmware instructions. Processors of the logic subsystem may be single-core or multi-core, and the instructions executed thereon may be configured for sequential, parallel, and/or distributed processing. Individual components of the logic subsystem optionally may be distributed among two or more separate devices, which may be remotely located and/or configured for coordinated processing. Aspects of the logic subsystem may be virtualized and executed by remotely-accessible, networked computing devices configured in a cloud-computing configuration.

Storage subsystem 1304 includes one or more physical devices configured to temporarily and/or permanently hold computer information such as data and instructions executable by the logic subsystem. When the storage subsystem includes two or more devices, the devices may be collocated and/or remotely located. Storage subsystem 1304 may include volatile, nonvolatile, dynamic, static, read/write, read-only, random-access, sequential-access, location-addressable, file-addressable, and/or content-addressable devices. Storage subsystem 1304 may include removable and/or built-in devices. When the logic subsystem executes instructions, the state of storage subsystem 1304 may be transformed—e.g., to hold different data.

Aspects of logic subsystem 1302 and storage subsystem 1304 may be integrated together into one or more hardware-logic components. Such hardware-logic components may include program- and application-specific integrated circuits (PASIC/ASICs), program- and application-specific standard products (PSSP/ASSPs), system-on-a-chip (SOC), and complex programmable logic devices (CPLDs), for example.

The logic subsystem and the storage subsystem may cooperate to instantiate one or more logic machines. As used herein, the terms “machine” (e.g., posture assessment machine, posture correction machine) and machine learning model (e.g., user interaction model, user productivity model, camera usage model, and location model) are used to collectively refer to the combination of hardware, firmware, software, instructions, and/or any other components cooperating to provide computer functionality. In other words, “machines” and “models” are never abstract ideas and always have a tangible form. A machine and/or model may be instantiated by a single computing device, or a machine may include two or more sub-components instantiated by two or more different computing devices. In some implementations a machine includes a local component (e.g., software application executed by a computer processor) cooperating with a remote component (e.g., cloud computing service provided by a network of server computers). The software and/or other instructions that give a particular machine its functionality may optionally be saved as one or more unexecuted modules on one or more suitable storage devices.

Machines may be implemented using any suitable combination of state-of-the-art and/or future machine learning (ML), artificial intelligence (AI), and/or natural language processing (NLP) techniques. Non-limiting examples of techniques that may be incorporated in an implementation of one or more machines include support vector machines, multi-layer neural networks, convolutional neural networks (e.g., including spatial convolutional networks for processing images and/or videos, temporal convolutional neural networks for processing audio signals and/or natural language sentences, and/or any other suitable convolutional neural networks configured to convolve and pool features across one or more temporal and/or spatial dimensions), recurrent neural networks (e.g., long short-term memory networks), Transformer-based machine learning models (e.g., Bidirectional Encoder Representations from Transformers), associative memories (e.g., lookup tables, hash tables, Bloom Filters, Neural Turing Machine and/or Neural Random Access Memory), word embedding models (e.g., GloVe or Word2Vec), unsupervised spatial and/or clustering methods (e.g., nearest neighbor algorithms, topological data analysis, and/or k-means clustering), graphical models (e.g., (hidden) Markov models, Markov random fields, (hidden) conditional random fields, and/or AI knowledge bases), and/or natural language processing techniques (e.g., tokenization, stemming, constituency and/or dependency parsing, and/or intent recognition, segmental models, and/or super-segmental models (e.g., hidden dynamic models)).

In some examples, the methods and processes described herein may be implemented using one or more differentiable functions, wherein a gradient of the differentiable functions may be calculated and/or estimated with regard to inputs and/or outputs of the differentiable functions (e.g., with regard to training data, and/or with regard to an objective function). Such methods and processes may be at least partially determined by a set of trainable parameters. Accordingly, the trainable parameters for a particular method or process may be adjusted through any suitable training procedure, in order to continually improve functioning of the method or process.

Non-limiting examples of training procedures for adjusting trainable parameters include supervised training (e.g., using gradient descent or any other suitable optimization method), zero-shot, few-shot, unsupervised learning methods (e.g., classification based at least on classes derived from unsupervised clustering methods), reinforcement learning (e.g., deep Q learning based at least on feedback) and/or generative adversarial neural network training methods, belief propagation, RANSAC (random sample consensus), contextual bandit methods, maximum likelihood methods, and/or expectation maximization. In some examples, a plurality of methods, processes, and/or components of systems described herein may be trained simultaneously with regard to an objective function measuring performance of collective functioning of the plurality of components (e.g., with regard to reinforcement feedback and/or with regard to labelled training data). Simultaneously training the plurality of methods, processes, and/or components may improve such collective functioning. In some examples, one or more methods, processes, and/or components may be trained independently of other components (e.g., offline training on historical data).

Language models may utilize vocabulary features to guide sampling/searching for words for recognition of speech. For example, a language model may be at least partially defined by a statistical distribution of words or other vocabulary features. For example, a language model may be defined by a statistical distribution of n-grams, defining transition probabilities between candidate words according to vocabulary statistics. The language model may be further based at least on any other appropriate statistical features, and/or results of processing the statistical features with one or more machine learning and/or statistical algorithms (e.g., confidence values resulting from such processing). In some examples, a statistical model may constrain what words may be recognized for an audio signal, e.g., based at least on an assumption that words in the audio signal come from a particular vocabulary.

Alternately or additionally, the language model may be based at least on one or more neural networks previously trained to represent audio inputs and words in a shared latent space, e.g., a vector space learned by one or more audio and/or word models (e.g., wav2letter and/or word2vec). Accordingly, finding a candidate word may include searching the shared latent space based at least on a vector encoded by the audio model for an audio input, in order to find a candidate word vector for decoding with the word model. The shared latent space may be utilized to assess, for one or more candidate words, a confidence that the candidate word is featured in the speech audio.

The language model may be used in conjunction with an acoustical model configured to assess, for a candidate word and an audio signal, a confidence that the candidate word is included in speech audio in the audio signal based at least on acoustical features of the word (e.g., mel-frequency cepstral coefficients, formants, etc.). Optionally, in some examples, the language model may incorporate the acoustical model (e.g., assessment and/or training of the language model may be based at least on the acoustical model). The acoustical model defines a mapping between acoustic signals and basic sound units such as phonemes, e.g., based at least on labelled speech audio. The acoustical model may be based at least on any suitable combination of state-of-the-art or future machine learning (ML) and/or artificial intelligence (AI) models, for example: deep neural networks (e.g., long short-term memory, temporal convolutional neural network, restricted Boltzmann machine, deep belief network), hidden Markov models (HMM), conditional random fields (CRF) and/or Markov random fields, Gaussian mixture models, and/or other graphical models (e.g., deep Bayesian network). Audio signals to be processed with the acoustic model may be pre-processed in any suitable manner, e.g., encoding at any suitable sampling rate, Fourier transform, band-pass filters, etc. The acoustical model may be trained to recognize the mapping between acoustic signals and sound units based at least on training with labelled audio data. For example, the acoustical model may be trained based at least on labelled audio data comprising speech audio and corrected text, in order to learn the mapping between the speech audio signals and sound units denoted by the corrected text. Accordingly, the acoustical model may be continually improved to improve its utility for correctly recognizing speech audio.

In some examples, in addition to statistical models, neural networks, and/or acoustical models, the language model may incorporate any suitable graphical model, e.g., a hidden Markov model (HMM) or a conditional random field (CRF). The graphical model may utilize statistical features (e.g., transition probabilities) and/or confidence values to determine a probability of recognizing a word, given the speech audio and/or other words recognized so far. Accordingly, the graphical model may utilize the statistical features, previously trained machine learning models, and/or acoustical models to define transition probabilities between states represented in the graphical model.

When included, display subsystem 1306 may be used to present a visual representation of data held by storage subsystem 1304. This visual representation may take the form of a graphical user interface (GUI). Display subsystem 1306 may include one or more display devices utilizing virtually any type of technology. In some implementations, display subsystem may include one or more virtual-, augmented-, or mixed reality displays.

When included, input subsystem 1308 may comprise or interface with one or more input devices. An input device may include a sensor device or a user input device. Examples of user input devices include a keyboard, mouse, touch screen, or game controller. In some embodiments, the input subsystem may comprise or interface with selected natural user input (NUI) componentry. Such componentry may be integrated or peripheral, and the transduction and/or processing of input actions may be handled on- or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition.

When included, communication subsystem 1310 may be configured to communicatively couple computing system 1300 with one or more other computing devices. Communication subsystem 1310 may include wired and/or wireless communication devices compatible with one or more different communication protocols. The communication subsystem may be configured for communication via personal-, local- and/or wide-area networks.

When the methods and processes described herein incorporate ML and/or AI components, the ML and/or AI components may make decisions based at least partially on training of the components with regard to training data. Accordingly, the ML and/or AI components can and should be trained on diverse, representative datasets that include sufficient relevant data for diverse users and/or populations of users. In particular, training data sets should be inclusive with regard to different human individuals and groups, so that as ML and/or AI components are trained, their performance is improved with regard to the user experience of the users and/or populations of users.

ML and/or AI components may additionally be trained to make decisions so as to minimize potential bias towards human individuals and/or groups. For example, when AI systems are used to assess any qualitative and/or quantitative information about human individuals or groups, they may be trained so as to be invariant to differences between the individuals or groups that are not intended to be measured by the qualitative and/or quantitative assessment, e.g., so that any decisions are not influenced in an unintended fashion by differences among individuals and groups.

ML and/or AI components may be designed to provide context as to how they operate, so that implementers of ML and/or AI systems can be accountable for decisions/assessments made by the systems. For example, ML and/or AI systems may be configured for replicable behavior, e.g., when they make pseudo-random decisions, random seeds may be used and recorded to enable replicating the decisions later. As another example, data used for training and/or testing ML and/or AI systems may be curated and maintained to facilitate future investigation of the behavior of the ML and/or AI systems with regard to the data. Furthermore, ML and/or AI systems may be continually monitored to identify potential bias, errors, and/or unintended outcomes.

This disclosure is presented by way of example and with reference to the associated drawing figures. Components, process steps, and other elements that may be substantially the same in one or more of the figures are identified coordinately and are described with minimal repetition. It will be noted, however, that elements identified coordinately may also differ to some degree. It will be further noted that some figures may be schematic and not drawn to scale. The various drawing scales, aspect ratios, and numbers of components shown in the figures may be purposely distorted to make certain features or relationships easier to see.

In an example, a computing system, comprises a posture assessment machine configured to receive one or more posture assessment signals from one or more posture assessment sensors, and output an assessment of a human subject's posture based at least on the one or more posture assessment signals, the one or more posture assessment sensors including a camera, and the one or more posture assessment signals including one or more images of a human subject captured by the camera; and a posture correction machine configured to receive the one or more images of the human subject and the assessment of the human subject's posture, generate a virtual clone of the human subject having an improved posture relative to the human subject's posture, and generate a composite image including the virtual clone admixed with an image of the human subject. In this example and/or other examples, the composite image may include posture adjustment feedback indicating whether the human subject's posture approaches the improved posture of the virtual clone. In this example and/or other examples, the posture correction machine may be configured to receive, via user input from the human subject, a selection of a best-fit clone of a plurality of training clones visually presented to the human subject, and customize a posture of the virtual clone based at least on the best-fit clone. In this example and/or other examples, the posture assessment machine may be configured to receive a plurality of posture assessment signals from the one or more posture assessment sensors over a posture tracking duration, progressively update the assessment of the human subject's posture over the posture tracking duration based at least on the plurality of posture assessment signals, and the posture correction machine may be configured to generate a posture assessment notification based at least on the progressively updated assessments of the human subject's posture over the posture tracking duration, the posture assessment notification visually summarizing how the human subject's posture changed over the posture tracking duration. In this example and/or other examples, the plurality of posture assessment signals may include a plurality of images of the human subject captured by the camera over the posture tracking duration, and the posture assessment notification may be derived from the plurality of images of the human subject captured over the posture tracking duration. In this example and/or other examples, the posture assessment notification may include a visual representation of different time intervals during the posture tracking duration where the posture assessment machine outputs assessments of the human subject's posture. In this example and/or other examples, the posture assessment notification may include context tags indicating different activities the human subject was involved in during the different time intervals. In this example and/or other examples, the posture assessment notification may further include a benefits notification indicating posture improving benefits that are currently available for the human subject. In this example and/or other examples, the one or more posture assessment sensors may include a microphone, the one or more posture assessment signals may include an audio signal corresponding to the human subject's voice acquired by the microphone, and the posture assessment machine may be configured to output the assessment of the human subject's posture further based at least on the audio signal. In this example and/or other examples, the one or more posture assessment signals may include one or more user-state metrics output from one or more trained machine-learning models. In this example and/or other examples, the one or more user-state metrics may include a user interaction metric indicating a level of user interaction based at least on user communication information generated by one or more productivity application programs and/or one or more personal communication application programs. In this example and/or other examples, the one or more user-state metrics may include a user productivity metric indicating a level of user productivity based at least on computing information generated by one or more productivity application programs. In this example and/or other examples, the one or more user-state metrics may include a camera usage metric indicating a level of camera usage during user interactions facilitated by one or more personal communication application programs.

In another example, a computer-implemented method comprises receiving one or more posture assessment signals from one or more posture assessment sensors including a camera, the one or more posture assessment signals including one or more images of a human subject captured by the camera, generating, via a posture assessment machine, an assessment of a human subject's posture based at least on the one or more posture assessment signals, generating, via a posture correction machine of the human subject, a virtual clone of the human subject having an improved posture relative to the human subject's posture, and generating, via the posture correction machine, a composite image including the virtual clone admixed with an image of the human subject. In this example and/or other examples, the composite image may include posture adjustment feedback indicating whether the human subject's posture approaches the improved posture of the virtual clone. In this example and/or other examples, the computer-implemented method may further comprise receiving, via user input from the human subject, a selection of a best-fit clone of a plurality of training clones visually presented to the human subject, and customizing, via the posture correction machine, a posture of the virtual clone based at least on the best-fit clone. In this example and/or other examples, the computer-implemented method may further comprise receiving a plurality of posture assessment signals from the one or more posture assessment sensors over a posture tracking duration, progressively updating, via the posture assessment machine, the assessment of the human subject's posture over the posture tracking duration based at least on the plurality of posture assessment signals, and generating, via the posture correction machine, a posture assessment notification based at least on the progressively updated assessments of the human subject's posture over the posture tracking duration, the posture assessment notification visually summarizing how the human subject's posture changed over the posture tracking duration.

In yet another example, a computer-implemented method comprises receiving a plurality of posture assessment signals from one or more posture assessment sensors over a posture tracking duration, progressively updating, via a posture assessment machine, an assessment of a human subject's posture over the posture tracking duration based at least on the plurality of posture assessment signals, generating, via a posture correction machine, a posture assessment notification based at least on the progressively updated assessments of the human subject's posture over the posture tracking duration, the posture assessment notification visually summarizing how the human subject's posture changed over the posture tracking duration. In this example and/or other examples, the one or more posture assessment sensors may include a camera, and the plurality of posture assessment signals may include a plurality of images of the human subject captured by the camera over the posture tracking duration, and the posture assessment notification may be derived from the plurality of images of the human subject captured over the posture tracking duration. In this example and/or other examples, the posture assessment notification may include a visual representation of different time intervals during the posture tracking duration where the posture assessment machine outputs assessments of the human subject's posture and associated context tags indicating different activities the human subject was involved in during the different time intervals.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A computing system, comprising:
a posture assessment machine configured to:
receive one or more posture assessment signals from one or more posture assessment sensors, and
output an assessment of a human subject's posture based at least on the one or more posture assessment signals, the one or more posture assessment sensors including a camera, and the one or more posture assessment signals including one or more images of a human subject captured by the camera; and
a posture correction machine configured to:
receive the one or more images of the human subject and the assessment of the human subject's posture,
generate a virtual clone of the human subject having an improved posture relative to the human subject's posture, and
generate a composite image including the virtual clone admixed with an image of the human subject.

2. The computing system of claim 1, wherein the composite image includes posture adjustment feedback indicating whether the human subject's posture approaches the improved posture of the virtual clone.

3. The computing system of claim 1, wherein the posture correction machine is configured to:
receive, via user input from the human subject, a selection of a best-fit clone of a plurality of training clones visually presented to the human subject, and
customize a posture of the virtual clone based at least on the best-fit clone.

4. The computing system of claim 1, wherein the posture assessment machine is configured to:
receive a plurality of posture assessment signals from the one or more posture assessment sensors over a posture tracking duration,
progressively update the assessment of the human subject's posture over the posture tracking duration based at least on the plurality of posture assessment signals, and
wherein the posture correction machine is configured to:
generate a posture assessment notification based at least on the progressively updated assessments of the human subject's posture over the posture tracking duration, the posture assessment notification visually summarizing how the human subject's posture changed over the posture tracking duration.

5. The computing system of claim 4, wherein the plurality of posture assessment signals includes a plurality of images of the human subject captured by the camera over the posture tracking duration, and wherein the posture assessment notification is derived from the plurality of images of the human subject captured over the posture tracking duration.

6. The computing system of claim 4, wherein the posture assessment notification includes a visual representation of different time intervals during the posture tracking duration where the posture assessment machine outputs assessments of the human subject's posture.

7. The computing system of claim 6, wherein the posture assessment notification includes context tags indicating different activities the human subject was involved in during the different time intervals.

8. The computing system of claim 4, wherein the posture assessment notification further includes a benefits notification indicating posture improving benefits that are currently available for the human subject.

9. The computing system of claim 1, wherein the one or more posture assessment sensors includes a microphone, wherein the one or more posture assessment signals includes an audio signal corresponding to the human subject's voice acquired by the microphone, and wherein the posture assessment machine is configured to output the assessment of the human subject's posture further based at least on the audio signal.

10. The computing system of claim 1, wherein the one or more posture assessment signals includes one or more user-state metrics output from one or more trained machine-learning models.

11. The computing system of claim 10, wherein the one or more user-state metrics includes a user interaction metric indicating a level of user interaction based at least on user communication information generated by one or more productivity application programs and/or one or more personal communication application programs.

12. The computing system of claim 10, wherein the one or more user-state metrics includes a user productivity metric indicating a level of user productivity based at least on computing information generated by one or more productivity application programs.

13. The computing system of claim 10, wherein the one or more user-state metrics includes a camera usage metric indicating a level of camera usage during user interactions facilitated by one or more personal communication application programs.

14. A computer-implemented method, comprising:
receiving one or more posture assessment signals from one or more posture assessment sensors including a camera, the one or more posture assessment signals including one or more images of a human subject captured by the camera;
generating, via a posture assessment machine, an assessment of a human subject's posture based at least on the one or more posture assessment signals;
generating, via a posture correction machine of the human subject, a virtual clone of the human subject having an improved posture relative to the human subject's posture; and
generating, via the posture correction machine, a composite image including the virtual clone admixed with an image of the human subject.

15. The computer-implemented method of claim 14, wherein the composite image includes posture adjustment feedback indicating whether the human subject's posture approaches the improved posture of the virtual clone.

16. The computer-implemented method of claim 14, further comprising:
receiving, via user input from the human subject, a selection of a best-fit clone of a plurality of training clones visually presented to the human subject; and
customizing, via the posture correction machine, a posture of the virtual clone based at least on the best-fit clone.

17. The computer-implemented method of claim 14, further comprising:
receiving a plurality of posture assessment signals from the one or more posture assessment sensors over a posture tracking duration;
progressively updating, via the posture assessment machine, the assessment of the human subject's posture over the posture tracking duration based at least on the plurality of posture assessment signals; and
generating, via the posture correction machine, a posture assessment notification based at least on the progressively updated assessments of the human subject's posture over the posture tracking duration, the posture assessment notification visually summarizing how the human subject's posture changed over the posture tracking duration.

18. A computer-implemented method, comprising:
receiving a plurality of posture assessment signals from one or more posture assessment sensors over a posture tracking duration;
progressively updating, via a posture assessment machine, an assessment of a human subject's posture over the posture tracking duration based at least on the plurality of posture assessment signals; and
generating, via a posture correction machine, a posture assessment notification based at least on the progressively updated assessments of the human subject's posture over the posture tracking duration, the posture assessment notification visually summarizing how the human subject's posture changed over the posture tracking duration.

19. The computer-implemented method of claim 18, wherein the one or more posture assessment sensors includes a camera, and wherein the plurality of posture assessment signals includes a plurality of images of the human subject captured by the camera over the posture tracking duration, and wherein the posture assessment notification is derived from the plurality of images of the human subject captured over the posture tracking duration.

20. The computer-implemented method of claim 18, wherein the posture assessment notification includes a visual representation of different time intervals during the posture tracking duration where the posture assessment machine outputs assessments of the human subject's posture and associated context tags indicating different activities the human subject was involved in during the different time intervals.
